(19) United States
(12) Patent Application Publication
(10) Pub. No.: US 2019/0380613 A1
(43) Pub. Date: Dec. 19, 2019

(54) TEXTILE DEVICE FOR MEASURING THE ELECTRO-PHYSIOLOGICAL ACTIVITY OF A SUBJECT

(71) Applicant: BIOSERENITY, Paris (FR)

(72) Inventors: Pierre PROT, Paris (FR); Pierre-Yves FROUIN, Paris (FR)

(73) Assignee: BIOSERENITY, Paris (FR)

(21) Appl. No.: 16/478,931

(22) PCT Filed: Jan. 19, 2018
(86) PCT No.: PCT/FR2018/050137

§ 371 Date: Jul. 18, 2019

(30) Foreign Application Priority Data
Jan. 19, 2017 (FR) 1750444

(51) Int. Cl. A61B 5/0478; A61B 5/00; D04B 1/12

(52) U.S. Cl. CPC A61B 5/0478; A61B 5/6804; D04B 1/12; A41D 13/1281; D10B 2403/02431; D10B 2509/00; A61B 2562/125

(57) ABSTRACT

A textile device for measuring the electrophysiological activity of a subject including at least one element for measuring the electrophysiological activity of the subject and a textile structure made by knitting including at least two conducting rows separated by a plurality of insulating rows; in which the at least one element for measuring of the electrophysiological activity the subject is connected to the surface of the textile structure intended to be in contact with the skin of the subject.

### FIELD OF INVENTION

The present invention relates to a textile device for measuring electrophysiological activity of a subject. In particular, the present invention relates to a textile device for measuring the electrophysiological activity of a subject comprising an electromagnetic shielding.

### BACKGROUND OF INVENTION

Electromagnetic interference between electronic devices has become increasingly important due to the increasing number of mobile and electronic devices in our environment.

Intelligent clothing for performing bioelectrical signal measurements requires passing signals of very low intensities, by means of electrical connections of significant length, driven by high contact impedances. These signals are then subject to a significant risk of electromagnetic or electrostatic disturbances. These disturbances can be varied. They can be caused by high frequency radio waves, electrostatic or low frequency disturbances, for example if another person passes near the sensors. The mains supply voltage is also likely to generate noise at 50 Hz or 60 Hz on the measured and transmitted signals.

These disturbances hinder the exploitation and interpretation of signals, whether they are made by technicians, doctors or even by computer algorithms. In the medical field, the doctor's expertise can enable him to distinguish different types of artifacts but in a very limited way. These disturbances can thus go as far as to distort a diagnosis, or to emit it with reservations. The subject cannot be diagnosed with certainty.

It is known from the prior art the international patent application WO 2013/126798 describing a helmet comprising electroencephalographic (EEG) electrodes on its inner surface intended to be in contact with the head of the subject. This helmet also includes on its outer surface, an electromagnetic shielding of these electrodes by the addition of a shielding surface. This shielding surface includes a conductive layer printed on a dielectric material in a particular pattern. The disadvantage of such a device is that it adds a thickness, a mass and additional rigidity to the existing helmet. Such a device is very rigid and the subject cannot wear such a device continuously over several days, especially during sleep phases.

It is known from the prior art the International patent application WO 00/11443 describing a flat electrode, notably for incorporation into a vehicle seat, comprising a conductive, flat textile material made of a metallically coated non-woven, woven or knitted fabric. To increase the electric conductivity of the metallic conductive textile material the randomly placed non-woven fibers or the intermeshing rows of woven or knitted fabric links are connected, preferably at defined intervals, in an electrically conductive manner by means a longitudinal contact element. Said flat electrode is a capacitive sensing electrode measuring a pressure. This variation of capacity allows the determination of the position or the movement of the user. Such a device does not include a shielding system against electromagnetic interference.

The object of the present invention is to develop an effective device for measuring the electrophysiological activity of a subject for shielding against electromagnetic interference, while also having good flexibility properties.

### SUMMARY

The present invention relates to a textile device for measuring the electrophysiological activity of a subject comprising:
at least one means for measuring the electrophysiological activity of the subject;
a textile structure made by knitting comprising at least two conducting rows separated by a plurality of insulating rows;
   in which the at least one means for measuring the electrophysiological activity of the subject is connected to the surface of the textile structure intended to be in contact with the skin of the subject.

According to one embodiment, the at least two conducting rows are substantially parallel to each other.

According to one embodiment, the knitted structure of the textile device further comprises at least one yarn-over-needle row substantially perpendicular to the at least two conducting rows; the at least one yarn-over-needle row is conductive and electrically connects the at least two conducting rows to each other.

According to one embodiment, the conductive rows consist of an insulating textile wire covered by a conductive surface, preferably a metal surface, very preferably a silver surface.

According to one embodiment, the textile device further comprising an electronic device comprising a ground, and wherein the at least two conducting rows are connected to said ground of the electronic device.

According to one embodiment, the at least one conductive yarn-over-needle row is metallic, preferably silver or consists of an insulating textile wire covered with a conductive surface, preferably a metal surface such as silver.

According to one embodiment, the means for measuring the electrophysiological activity of the subject comprises at least one electrode, a printed circuit, and an electrical connection means connecting the at least one electrode to the printed circuit.

According to one embodiment, the at least two conducting rows substantially parallel to each other are separated by a number of insulating rows which varies from 2 to 300.

The present invention further relates to a method of manufacturing an assembly comprising the following steps:
the knitting of a textile device according to the present invent;
cutting and sewing said textile device to the surface of a garment so that the surface of the device comprising the means for measuring the electro-physiological activity is in contact with the skin of the subject wearing said garment.

The present invention further relates to a garment comprising one or more textile devices according to any one of embodiment described hereabove, or manufactured by the method of manufacturing described hereabove.

### Definitions

In the present invention, the following terms have the following meanings:
“Row”: refers to a yarn in a knitted structure. A row is a yarn forming meanders or stitches of the knitted structure.
“Mesh”: refers to a hole formed by the loops of the rows.
“Average path of a row”: refers to the average direction of the axis of a row. The average path can be drawn by connecting each mesh of the same row. An average path is the direction of a row of mesh to mesh.
“Substantially perpendicular” should be understood as perpendicular to plus or minus 25°, preferentially plus or minus 10°, and very preferentially plus or minus 5°. The term “substantially perpendicular”, used with respect to one or more rows forming part of a knitted structure, should be interpreted as substantially perpendicular to the average path formed by said rows.
“Substantially parallel rows” shall be understood as two rows separated by the same number of rows over a given distance, preferably on at least 1 cm, 2 cm, 3 cm, 4 cm, 5 cm, 6 cm, 7 cm, 8 cm, 9 cm or 10 cm.
“Plurality” should be understood as “at least two”.
“Garment” includes any textile that can be worn by a subject, such as an animal or a human being, and may further include a hat.
“Smart clothing” means any textile capable of being worn by a subject comprising at least one conductive area configured to transmit or receive an electrical signal.
By “intended to be in contact with the skin of a subject”, one also understands the skull of the subject, whatever its hairs.

### DETAILED DESCRIPTION

The present invention aims at manufacturing a textile device for measuring electrophysiological activity of a subject configured to electromagnetically shield against external disturbances. The electromagnetic shielding of this device is achieved by the knitting of a textile structure comprising at least two conducting rows separated by a plurality of insulating rows. The textile device comprises a means for measuring the electrophysiological activity of a subject. The means of measuring activity electrophysiological the subject is connected to the surface of the textile structure intended to be in contact with the skin of the subject. In this way, the signal received and transmitted by the at least one means for measuring the electrophysiological activity of a subject is protected from any electromagnetic or electrostatic disturbances experienced by the subject in his environment. In one embodiment, the means for measuring is a means for measuring the electrical activity of a subject.

As a result, the device according to the present invention essentially comprises a textile structure and a means for measuring.

According to the present invention, the conducting rows form a conductive network knitted in the textile structure.

According to one embodiment, the textile structure is a knitted structure, comprising conducting rows and insulating rows. A knitted structure is a three-dimensional structure. A knitted structure allows for meshes of greater thickness, lighter, and therefore more comfortable for the subject than the mesh of a woven structure. The knitting also makes it possible to be able to select the places to be shielded or not along the garment. In addition, the three-dimensional structure of a knitted structure is particularly advantageous for the purpose of the present invention. Indeed, this knitted structure allows the attenuation of electromagnetic signals by the thickness of the textile structure, thus improving the electromagnetic shielding of the at least one means for measuring the electrophysiological activity of the subject.

In one embodiment, said knitted fabric structure comprises a plurality of knit thicknesses.

According to one embodiment, the textile device according comprises a conductive network (also called Faraday grating), composed of knitted conductive rows. The textile device consisting of a continuous and uniform Faraday network is a shield against electromagnetic radiation.

The textile structure comprising a network of electrically connected conducting wires acts as a Faraday cage of the means for measuring located on the surface of the garment intended to be in contact with the skin of the subject. The textile structure is therefore able to operate an electromagnetic shielding of the means for measuring the electrophysiological activity of the subject.

The yarn of a knitted fabric follows a meander path, forming loops (or meshes) successively below and above an average path. According to one embodiment, the textile structure obtained by knitting comprises at least two medium paths made with at least two conductive wires separated by a plurality of medium paths made of insulating wire.

According to one embodiment, the conductive rows are not perpendicular to the knitted fabric.

As illustrated in FIG. 1, the textile device 1 according to the present invention comprises a textile structure 10 which comprises at least two conductive rows 11 separated by a plurality of insulating rows 12. The at least two conductive rows 11 are separated by at least two insulating rows 12. In one embodiment, the at least two conductive rows 11 are separated by at least 3 insulating rows 12. In another embodiment, the at least two conductive rows 11 are separated by at least 4, 5 rows, 6, 7, 8, 9, 10, 15, 20, 25, 30, 40, 50, 60, 70, 80, 90, 100, 200 or 300 insulating rows 12. In one embodiment, the at least two conducting rows are separated by a number of insulating rows 12 which varies from 2 to 300, from 3 to 100.

For example, the number of insulating rows knitted between two conducting rows may range between 5 and 50. The electromagnetic shielding effect of the Faraday cage is then optimal when the two substantially parallel conducting rows are separated by a number of insulating rows which varies between 5 and 50 insulating rows.

In one embodiment, the textile device also comprises an electronic device comprising a ground. The network of conducting wires or the at least two conducting rows are connected to said ground of the electronic device. The connection to the ground of the network of conducting wires makes it possible to keep the potential of the Faraday cage fixed and to protect the inside of the cage from electromagnetic disturbances. In one embodiment, the ground is the subject carrying the textile device. In this embodiment, the conducting rows comprise a portion opening on the surface of the textile structure intended to be in contact with the skin of the subject.

In one embodiment, the at least two conducting rows are connected to a ground. In one embodiment, the textile device according to the present invention comprises at least one connecting means adapted to connect the network formed by the conductive rows and the conductive yarn-over-needle row to a subject carrying said textile device.

In one embodiment, the at least two conducting rows of the textile structure are substantially parallel to one another.

In one embodiment, the at least two conducting rows are substantially parallel to each other and the textile structure also comprises yarn-over-needle row substantially perpendicular to the average paths of the at least two conducting rows. The yarn-over-needle row of the textile structure are made with conductive threads.

Yarn over needle is a knitting technic known to those skilled in the art. Its role, usually decorative, allows to link two meshes on different paths. Here, the interest is to make an electrical connection between the at least two conducting rows to facilitate the homogenization of the potential.

In an embodiment not shown, the use of conductive yarn-over-needle rows as connection means between two conducting rows has several advantages. At first, it allows the conductive rows not to come into direct contact. The conductive rows thus strictly follow the structure of the knit and do not need to be diverted to make contact. In addition, a conductive yarn-over-needle row is an integral part of the textile structure. It is therefore much more resistant to wear than the standard connection means.

In one embodiment, each of the at least two conductive rows substantially parallel to each other are connected to ground.

In one embodiment, the textile structure comprises at least two conductive discarded rows substantially perpendicular to the at least two conducting rows and the two conductive yarn-over-needle rows are spaced apart by a length of between 0.1 cm and 50 cm, between 0.1 cm. and 30 cm, between 0.5 cm and 10 cm or between 0.5 cm 5 cm.

In one embodiment, the textile structure comprises a plurality of yarn-over-needle rows disposed evenly spaced to form a network with the conducting rows substantially parallel to each other and the yarn-over-needle rows are regularly spaced at a distance between 1 and 100 mm, between 5 and 75 mm, between 8 and 50 mm, or between 10 and 40 mm.

In a first embodiment, the conductive wires used to make the conductive rows are wires made of a conductive material. Preferably, said conductive wires are metal wires, such as silver wires.

In a second embodiment, the conductive wires used to produce the conductive rows are wires comprising an insulating textile wire covered with a conductive surface. Said wires preferably comprise an insulating textile wire covered with a metal surface, and very preferably a textile wire covered with a silver surface. Preferably, the insulating textile wire covered with a conductive surface is made of polyamide. Very preferably, the conductive wire therefore comprises a polyamide insulating wire covered with a silver surface or a silver sheath. This embodiment is particularly effective for obtaining a textile structure comprising both good electromagnetic shielding properties and good flexibility since the conductive yarns retain the lightness and flexibility of the insulating wires, for example polyamide yarns, unlike metal wires. This embodiment makes it possible to obtain wires, and therefore a textile structure that is more flexible, less thick, and less rigid than a similar structure obtained with wires entirely made of metallic material or silver. Furthermore, the integration of these wires into a knit structure is also facilitated.

In a third embodiment, said conducting wires used to produce the conducting rows are a combination of the first two embodiments mentioned above.

In the embodiment where the textile structure comprises at least one yarn-over-needle row substantially perpendicular to the average paths of the at least two conducting rows, the said at least one yarn-over-needle row is made according to one of the three embodiments mentioned above. In a preferred embodiment, said at least one conductive yarn-over-needle row consists of an insulating textile wire covered with a conductive surface, preferably a metal surface such as silver.

In one embodiment, the at least two conducting rows and the at least one yarn-over-needle row are not made of metal wire. In one embodiment, the at least two conducting rows or the at least one yarn-over-needle row are not made of wire comprising a metal core.

In one embodiment, the at least two conducting rows or the at least one yarn-over-needle row are not made of metal wire. In one embodiment, the at least two conducting rows and the at least one yarn-over-needle row are not made of wire comprising a metal core.

In one embodiment, the length over which the substantially parallel rows are separated by the same number of rows is 1 cm, 2 cm, 3 cm, 4 cm, 5 cm, 6 cm, 7 cm, 8 cm, 9 cm or 10 cm.

According to one embodiment, the textile device for measuring the electro-physiological activity comprises at least one means for measuring the electrophysiological activity of the subject. According to one embodiment, the at least one means of measuring electrophysiological activity the subject is connected to the surface of the textile structure intended to be in contact with the skin of the subject. In this way, the at least two conducting rows separated by a plurality of insulating rows, forming a Faraday network, are placed so as to protect from the electromagnetic radiation the at least one means for measuring the electrophysiological activity of the subject.

In one embodiment, the textile device (1) comprises a means for measuring the electrophysiological activity and a textile structure comprising insulating wires and conductive wires forming an electromagnetic shielding structure.

In one embodiment illustrated in FIG. 2, the at least one means for measuring the electrophysiological activity of the subject is connected to the surface 13 of the textile structure 10 intended to be in contact with the skin of the subject. The conductive rows 11 make it possible to protect the measurement means 20 from external disturbances. In one embodiment, the means for measuring 20 is a textile electrode knitted in the textile structure 10. In one embodiment, the means for measuring 20 is connected to the textile structure 10 by means of connection means. These connection means may be magnetic means and/or mechanical means.

In one embodiment, the at least one means for measuring the activity electrophysiological of the subject's may also be any means of measuring a biophysiological parameter.

In one embodiment, the at least one means for measuring the electrophysiological activity of the subject is a sensor (preferably an electrode) for measuring EEG activity, preferably an EEG electrode. The at least one means for measuring the electrophysiological activity of the subject may also be a sensor (preferably an electrode) configured to measure the electrophysiological activity of the cardiac muscle or nerve function and/or peripheral muscles, such as, for example, masticatory muscles.

In a particular embodiment illustrated in FIG. 3, the at least one means for measuring the electrophysiological activity 20 of the subject comprises at least one electrode 21, a printed circuit 22, and an electrical connection means 23 connecting the at least one electrode 21 to the printed circuit 22. Preferably the at least one electrode is a surface electrode. In this embodiment, the assembly of the means for measuring (electrode 21, printed circuit 22 and connection means 23) is protected from disturbances by the Faraday network formed by the textile structure 10 according to the present invention. In one embodiment, the connection means 23 is a conductive thread of the textile structure 10.

In one embodiment, the subject is a human being.

In one embodiment, the subject is an animal.

The invention also relates to a method for manufacturing an assembly comprising:
knitting a textile device, said textile device comprising:
   at least one means for measuring the electrophysiological activity of a subject;
   at least two conducting rows separated by a plurality of insulating rows; and
      wherein the at least one means for measuring the electrophysiological activity of the subject is connected to the surface of the textile structure intended to be in contact with the skin of the subject;
cutting and sewing said textile device so as to give said textile device the shape adapted to be part of a garment adapted to be worn by a subject.

The invention also relates to a method for manufacturing an assembly comprising:
knitting a textile device, said textile device comprising:
   at least one means for measuring the electrophysiological activity of a subject;
   at least two conducting rows separated by a plurality of insulating rows; and
      wherein the at least one means for measuring the electrophysiological activity of the subject is connected to the surface of the textile structure intended to be in contact with the skin of the subject;
cutting and sewing said textile device onto the surface of a garment so that the surface of the device comprising the means for measuring the electro-physiological activity is in contact with the skin of the subject wearing said garment.

The invention also relates to a garment made with one or more textile devices according to the present invention or by the manufacturing method according to the present invention. The garment thus realized forms an electromagnetic shield for the means of measuring the electro-physiological activity of the subject who carries it thanks to the network of conductive wires forming a Faraday cage. In a preferred embodiment, the garment is a hat or helmet configured to measure the electrophysiological activity of the brain.

The embodiment of a garment comprising the textile device according to the present invention has many advantages, particularly in terms of flexibility and performance

In one embodiment, the garment comprises a plurality of textile devices according to the present invention, connected to each other by textile structures that do not comprise conductive rows. Thus, the conducting rows are present only in the areas where there is present at least one means for measuring the activity of the electrophysiological activity subject, thereby greatly increasing the suppleness and flexibility of the garment, thereby improving the comfort of the subject, so that said subject can continue to wear said garment during his sleep phases.

### BRIEF DESCRIPTION OF THE DRAWINGS

FIG. 1 is a diagram illustrating the textile structure 10 comprising conductive rows 11 separated by insulating rows 12.

FIG. 2 is a sectional view of the device along the axis AA′ of FIG. 1.

FIG. 3 is a sectional view of the device along the axis AA′ of FIG. 1.

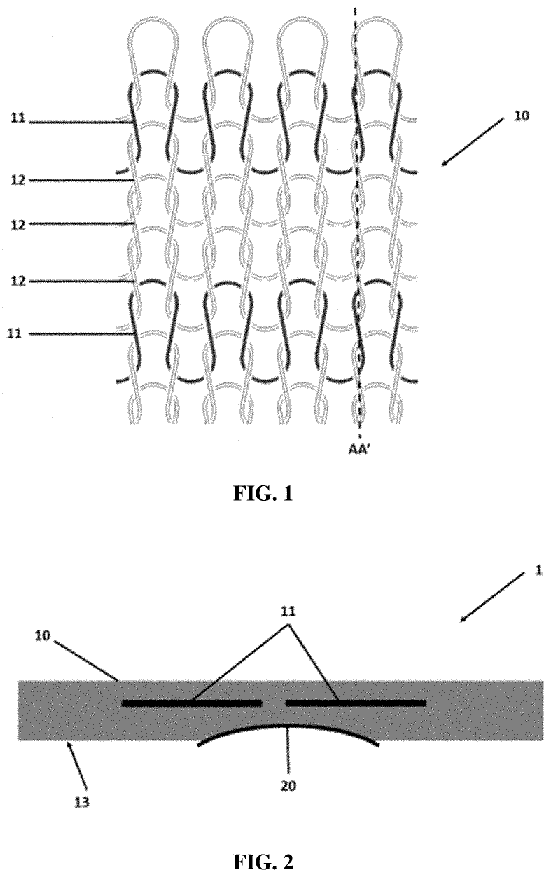

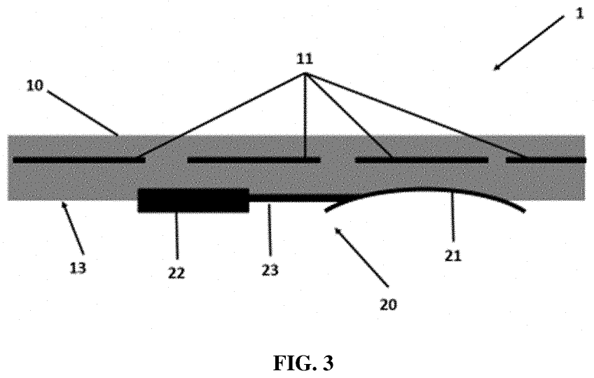

The present invention is not limited to the illustrated knitting structure but extends to all other possible structures and known to those skilled in the art. The illustrations are not to scale and the dimensions measured in the different figures cannot be used to interpret or limit the present invention.

### REFERENCES

1—Textile device;
10—Textile structure;
11—Conductive row;
12—Insulating rows;
13—Surface of the textile structure intended to be in contact with the skin of a subject;
20—Means of measuring the electrophysiological activity of a subject;
21—Surface electrode;
22—Printed circuit;
23—Electrical connection means.

1-10. (canceled)

11. A textile device for measuring the electrophysiological activity of a subject comprising:
(a) at least one measurement element for measuring the electrophysiological activity of the subject;
(b) a textile structure made by knitting comprising at least two conducting rows separated by a plurality of insulating rows;
in which the at least one measurement element for measuring the electrophysiological activity of the subject is connected to the surface of the textile structure intended to be in contact with the skin of the subject.

12. The textile device according to claim 11, wherein the at least two conducting rows are substantially parallel to each other.

13. The textile device according to claim 12, wherein the knitted structure of the textile device further comprises at least one yarn-over-needle row substantially perpendicular to the at least two conducting rows and wherein the at least one yarn-over-needle row is conductive and electrically connects the at least two conducting rows to each other.

14. The textile device according to claim 11, wherein the conductive rows consist of an insulating textile wire covered by a conductive surface, preferably a metal surface.

15. The textile device according to claim 14, wherein the conductive rows consist of an insulating textile wire covered by a silver surface.

16. The textile device according to claim 11, further comprising an electronic device comprising a ground, and wherein the at least two conducting rows are connected to said ground of the electronic device.

17. The textile device according to claim 13, wherein the at least one conductive yarn-over-needle row is metallic, preferably silver.

18. The textile device according to claim 13, wherein the at least one conductive yarn-over-needle row consists of an insulating textile wire covered with a conductive surface, preferably a metal surface.

19. The textile device according to claim 18, wherein the at least one conductive yarn-over-needle row consists of an insulating textile wire covered with silver.

20. The textile device according to claim 11, wherein the measurement element for measuring the electrophysiological activity of the subject comprises at least one electrode, a printed circuit, and an electrical connection element connecting the at least one electrode to the printed circuit.

21. The textile device according to claim 12, wherein the at least two conducting rows substantially parallel to each other are separated by a number of insulating rows which varies from 2 to 300.

22. A method of manufacturing an assembly comprising the following steps:
(a) knitting a textile device according to claim 11;
(b) cutting and sewing said textile device to the surface of a garment so that the surface of the device comprising the measurement element for measuring the electro-physiological activity is in contact with the skin of the subject wearing said garment.

23. Garment comprising one or more textile devices according to claim 11, or manufactured by a method of manufacturing an assembly comprising the following steps:
(a) knitting said one or more textile devices;
(b) cutting and sewing said one or more textile devices to the surface of a garment so that the surface of each of said one or more devices comprising the measurement element for measuring the electro-physiological activity is in contact with the skin of the subject wearing said garment.
